(19) United States
(12) Patent Application Publication
(10) Pub. No.: US 2022/0292521 A1
(43) Pub. Date: Sep. 15, 2022

(54) Computer-Assisted Method for Generating Training Data for a Neural Network for Predicting a Concentration of Pollutants

(71) Applicant: Siemens Aktiengesellschaft, München (DE)

(72) Inventors: Florian Ansgar Jaeger, Berlin (DE); Katrin Müller, Berlin (DE)

(73) Assignee: Siemens Aktiengesellschaft, München (DE)

(21) Appl. No.: 17/635,090

(22) PCT Filed: May 29, 2020
(86) PCT No.: PCT/EP2020/064986

§ 371 Date: Feb. 14, 2022

(30) Foreign Application Priority Data
Aug. 16, 2019 (DE) 10 2019 212.289.2

(51) Int. Cl. G06Q 30/00; G06N 3/04; G06N 3/08

(52) U.S. Cl. CPC G06Q 30/018; G06N 3/0472; G06N 3/08

(57) ABSTRACT

Various embodiments of the teachings herein include a computer-aided method for generating training data for a neural network used to determine a pollutant concentration from a pollutant emission. The method may include: providing a first series of the pollutant concentration with one reading above a defined threshold value; providing a second series for a physical measured variable related to the pollutant concentration; providing a model for a relationship between the two; computing a first value of the pollutant emission with the model using a value of the measured variable related to a value of the pollutant concentration; computing a second value of the pollutant emission with the model by numerically altering the measured value of the measured variable; and generating a synthetic measurement series as training data using an alteration of the value of the measured series, using the relative change in the computed values of the pollutant emissions.

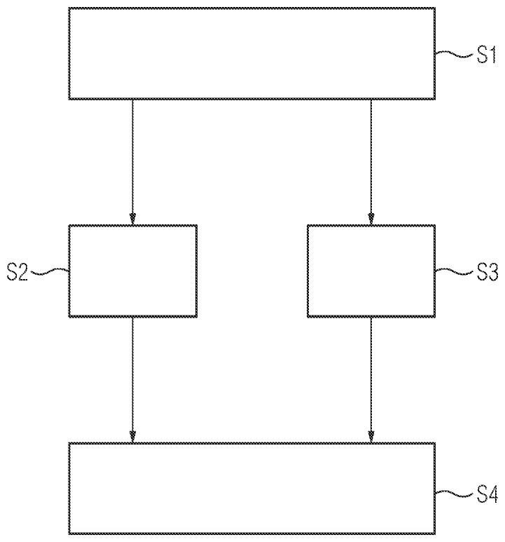

### CROSS-REFERENCE TO RELATED APPLICATIONS

This application is a U.S. National Stage Application of International Application No. PCT/EP2020/064986 filed May 29, 2020, which designates the United States of America, and claims priority to DE Application No. 10 2019 221 289.2 filed Aug. 16, 2019, the contents of which are hereby incorporated by reference in their entirety.

### TECHNICAL FIELD

The present disclosure relates to neural networks. Various embodiments include methods for generating training data for a neural network, methods for training a neural network, and/or methods for determining a pollutant concentration using a neural network.

### BACKGROUND

The pollutant burden, for example a nitrogen oxide concentration, can be above the permissible limit values within some German cities for specific periods of time. To guarantee adequate air quality, cities can take several measures, for example bans on driving. For these measures to be effective, however, it is necessary for them to be carried out even before the limit values are possibly exceeded. This requires a prediction (forecast) of the pollutant concentration that is reliable and as precise as possible.

In principle, a distinction is drawn between emissions (mass or mass per length per time dimension) and concentrations (mass per volume dimension). The emission is the emitted mass of a pollutant, for example from a road user within a time range, for example one hour. The emission may likewise relate to a length (road length, route length, etc.) and a time range, with the result that it comprises the dimension mass per length per time in this instance. The pollutant concentration is measured by a measurement station, for example, at a specific location within the town. In principle, the emissions and pollutant concentrations are time dependent.

The pollutant concentration is difficult to predict owing to the complexity of the processes, with the result that neural networks are typically used for this purpose. The fundamental method is split in two in this instance. First, the emission is computed by means of a model. The pollutant concentration is then determined by means of the neural network from the emission computed on a model basis.

This requires the neural network to be trained, that is to say that training data concerning the pollutant concentration are required. Symbolically, the neural network needs to use the training data to learn how the pollutant concentration results from the pollutant emission. Typically, the neural network is trained by using historical data of the pollutant concentration as training data. A neural network trained in this manner provides a good prediction in situations that occur frequently.

It is therefore possible for the average pollutant concentration to be predicted with sufficient accuracy by said prediction.

Events or situations involving a heavy burden are problematic because they are typically rare. As a result, only few data are available for training the neural network. This problem means that the prediction is poorer for the events involving a heavy burden that are actually of interest, that is to say for the rare events. Essentially two methods to improve the prediction for rare events such as these are known from the prior art.

First, the data or measurement series used for training can be weighted differently. By way of example, a historical event involving a heavy burden is used repeatedly. The disadvantage of this is that it impairs the prediction of the average burden. The actual problem that fewer measurement series or measurement data and hence training data are available for events involving a heavy burden therefore persists.

Second, the pollutant emission and pollutant concentration can be computed by a complete model-based approach. This is a large amount of effort, and also not all dependencies are known. The known methods therefore typically provide excessively low values for the pollutant concentration.

### SUMMARY

The teachings of the present disclosure provide improved training of a neural network provided for determining a pollutant concentration from a pollutant emission. As an example, some embodiments of the teachings herein include a computer aided method for generating training data for a neural network, wherein the neural network is designed to determine a pollutant concentration from at least one pollutant emission, including: providing at least one measurement series of the pollutant concentration containing at least one measured value that is above a defined threshold value; providing at least one measurement series for a physical measured variable related to the measured pollutant concentration, in particular a temperature, a wind speed and/or a traffic level; providing a model, wherein the model models a relationship between the measured variable and the pollutant emission; computing a first value E₀ of the pollutant emission by means of the model, this being accomplished by using at least one measured value of the measured variable that is related to a value C₀ of the provided measured pollutant concentration; computing a second value E₁ of the pollutant emission by means of the model, this being accomplished by numerically altering the measured value of the measured variable that is used for computing the first value E₀ of the pollutant emissions; and generating a synthetic measurement series as training data by means of an alteration Δ_{C} of the value C₀ of the provided measured measurement series of the pollutant concentrations, the alteration Δ_{C} being made by means of the relative change ΔE/ΔE₀ in the computed values of the pollutant emissions.

In some embodiments, the alteration Δ_{C} of the at least one value C₀ of the provided measurement series of the pollutant concentrations is additionally made by means of a traffic-related proportion α of the pollutant concentration.

In some embodiments, the alteration Δ_{C} of the at least one value C₀ of the provided measurement series of the pollutant concentration is made by means of ΔC/C₀=αΔE/E₀.

In some embodiments, a traffic-related proportion a in the range from 0.3 to 0.5 is used.

In some embodiments, a nitrogen oxide concentration is used as pollutant concentration and a nitrogen oxide emission is used as pollutant emission.

In some embodiments, the physical measured variable used is a temperature, a wind speed and/or a traffic level.

In some embodiments, the measurement series of the pollutant concentration and the measurement series of the measured variable were captured by means of a measurement station within a town.

In some embodiments, the model used is a domain based model.

As another example, some embodiments include a computer aided method for training a neural network, wherein the neural network is designed to determine a pollutant concentration from at least one pollutant emission, characterized in that a training dataset generated as described herein is used to train the neural network.

As another example, some embodiments include a computer aided method for determining a pollutant concentration by means of a neural network and by means of a model, wherein the neural network is designed to determine a pollutant concentration from at least one pollutant emission and is trained as described herein, wherein the model models a relationship between a physical measured variable, in particular a temperature, a wind speed and/or a traffic level, and the pollutant emission, including: computing a value of the pollutant emission by means of the model, this being accomplished by using at least one measured value of the measured variable; and determining the pollutant concentration from the computed value of the pollutant emission by means of the neural network.

### BRIEF DESCRIPTION OF THE DRAWINGS

Further advantages, features, and details of the teachings herein can be obtained from the exemplary embodiments described below and from the drawing. The single FIGURE of said drawing shows a schematic flowchart for a method incorporating teachings of the present disclosure. Elements that are of the same type, are equivalent or have the same effect may be provided with the same reference signs in the FIGURE.

The FIGURE shows a flowchart, or flow diagram, of a method incorporating teachings of the present disclosure.

### DETAILED DESCRIPTION

Some embodiments of the teachings herein include a computer aided method for generating training data for a neural network, wherein the neural network is designed to determine a pollutant concentration from at least one pollutant emission, comprising steps:
providing at least one measurement series of the pollutant concentration containing at least one measured value that is above a defined threshold value;
providing at least one measurement series for a physical measured variable related to the measured pollutant concentration, in particular a temperature, a wind speed and/or a traffic level;
providing a model, wherein the model models a relationship between the measured variable and the pollutant emission;
computing a first value E₀ of the pollutant emission by means of the model, this being accomplished by using at least one measured value of the measured variable that is related to a (original) value C₀ of the provided measured pollutant concentration;
computing a second value E₁ of the pollutant emission by means of the model, this being accomplished by numerically altering the measured value of the measured variable that is used for computing the first value E₀ of the pollutant emissions; and
generating a synthetic measurement series as training data by means of an alteration Δ_{C} of the value C₀ of the provided measured measurement series of the pollutant concentrations, the alteration Δ_{C} being made by means of the relative change ΔE/ΔE₀ in the computed values of the pollutant emissions.

The methods for generating training data provides data, or a time series, of the pollutant concentration, which can be used to train the neural network. The training can be effected by means of known methods, for example deep learning. The neural network (artificial neural network) is provided or designed in this case to determine a pollutant concentration from a pollutant emission. The pollutant emission, or the pollutant emissions, are computed by means of the model.

In some embodiments, a measurement series of a pollutant concentration is provided, wherein at least one value, or measured value, of the pollutant concentration is above the defined threshold value. In other words, a measurement series is provided that corresponds to a pollutant concentration that is high at at least one time and therefore to a heavy pollutant burden. A rare event of a heavy pollutant burden had therefore occurred.

The threshold value is typically defined by a limit value, for example 200 micrograms per cubic meter (μg/m³) for nitrogen oxide. The measurement series is a chronological sequence (continuous or discrete) of measured values of the pollutant concentration, for example in the unit μg/m³. The measurement series comprises one or more measured values, each measured value having been captured at a specific time. The time can likewise be a time range, with the result that a measured value was captured or determined for the time range. By way of example, a measured value of the pollutant concentration is determined for each hour, for example by one or more measurements. In other words, a measured value of the pollutant concentration is captured for each hour of a day, for example. The chronologically ordered sequence of these captured measured values then forms an exemplary measurement series of the pollutant concentration.

In some embodiments, at least one measurement series of a physical measured variable is provided. In this case, the measured variable is a physical variable, for example a temperature, a wind speed and/or a traffic level, or traffic density. The measured variable is related to the provided measured pollutant concentration, that is to say that a measured value of the pollutant concentration and a measured value of the measured variable are available for each time. There may be provision for multiple measured variables and corresponding measurement series.

By way of example, an average pollutant concentration and the average temperature, wind speed and/or traffic level prevailing for the respective average pollutant concentration, and therefore related, are captured for each hour of a day. In other words, at least two measured variables are captured over time, the pollutant concentration and the physical measured variable, for example the temperature, the wind speed and/or the traffic level that are or were prevailing for the measured pollutant concentration. The measured variable is significant because it or multiple measured variables, such as for example temperature, wind speed and/or traffic level, fundamentally influence the pollutant concentration, that is to say that the pollutant concentration is dependent on the one or more measured variables. As such, the pollutant concentration at a measurement station within a town can be decisively dependent on wind direction and/or traffic level.

In some embodiments, a model is provided, wherein the model models, or describes, a relationship (dependency) between the measured variable and the pollutant emission. The model can therefore be used to compute the pollutant emission, for example of a road user, on the basis of the measured variable, for example temperature, wind speed and/or traffic level. These models are typically complex and domain based. The model therefore comprises at least one input variable and at least one output variable, the input variable being the measured variable and the output variable being the pollutant emission.

In some embodiments, a first value E₀ of the pollutant emission is computed using the model. This is accomplished by using at least one measured value of the measured variable that is related to a value C₀ of the provided measured pollutant concentration. In other words, the value of the measured variable that is related to the value C₀ of the provided measured pollutant concentration, for example the value of the temperature that is related to the value of the pollutant concentration, is used as an input variable for the model. From this, the model then computes the first value E₀ of the pollutant emission. By way of example, temperature, wind speed and/or traffic level are put into the model as input variables, from which the model then computes the first pollutant emission E₀.

In some embodiments, a second value E₁ of the pollutant emission is computed by means of the model. This is accomplished by numerically altering the measured value of the measured variable that is used for computing the first value E₀ of the pollutant emissions. In other words, the second pollutant emission E₁ is computed for an altered value of the measured variable, for example for an altered value of temperature, wind speed and/or traffic level. The altered value of the measured variable or accordingly the altered measurement series of the measured variable is therefore put into the model as an input variable.

As a result, the second value of the pollutant emission E₁, or a second pollutant emission, or a second time series of the pollutant emission, is computed. With this in mind, the second value of the pollutant emission E₁ corresponds to a synthetic pollutant emission that would prevail for a corresponding altered value of the measured variable, for example for an altered temperature, an altered wind speed and/or an altered traffic level. In this case, it is advantageous to alter the value of the measured variable only slightly. By way of example, the relative change in the value of the measured variable is preferably less than 10 percent.

In some embodiments, a new, further or synthetic measurement series is generated, on which the training dataset is based. In other words, the training dataset comprises the new measurement series, the neural network being trainable by means of the new measurement series. The new measurement series is generated by means of an alteration Δ_{C} of the value C₀ of the provided measured measurement series of the pollutant concentrations, the alteration Δ_{C} being made by means of the relative change ΔE/E₀=(E₁−E₀)/E₀ in the computed values of the pollutant emissions. Since the new measurement series is based on the second (synthetically) computed value E₁ of the pollutant emission, and the second value E₁ is certainly not based on a measured value of the measured variable, the new, or further, measurement series of the pollutant concentration can likewise be referred to as a synthetic measurement series. In other words, the freshly generated measurement series for the provided measured measurement series has not been measured, but rather has been generated synthetically by means of the described method.

The teachings of the present disclosure therefore allow a plurality of synthetic measurement series of the pollutant concentration to be generated, which can be used to train the neural network, as with the originally measured measurement series of the pollutant concentration already. Since the original provided measured measurement series of the pollutant concentration corresponds to a rare event involving a heavy burden—this being guaranteed by the threshold value of the method—it is therefore possible to generate multiple measurement series of rare events involving a heavy burden synthetically. If the neural network is trained by means of these freshly generated synthetic measurement series, the prediction of the neural network for said rare events is improved without needing to expect a deterioration in the average response.

In other words, the teachings of the present disclosure allow the neural network to learn from a more extensive training dataset. This improves the prediction of the neural network with regard to the rare, but most relevant, events involving a heavy burden.

Furthermore, the integration of the prediction algorithm into already existing models is no more complex than the use of conventional algorithms for neural networks. This is the case because although these are improved, their structure remains unchanged. In other words, the teachings herein relate first of all to the training of the neural network, or the generation of a related training dataset, or extension of an already existing training dataset. In comparison with a weighting of measured values, it is likewise possible to produce a much better database. Compared to a complete model-based approach, the effort and data requirement are much lower. Furthermore, the model does not have to be operated online for a prediction, but rather merely needs to run for the specific and relevant events or scenarios for training the neural network. This may allow processing time to be saved. There can be provision for an online mode, however.

The teachings herein therefore allow a more accurate prediction for lower effort and reduced data requirements. The computer aided methods for training a neural network, wherein the neural network is designed to determine a pollutant concentration from at least one pollutant emission, include a training dataset generated according to the teachings herein and/or one of its configurations is used to train the neural network.

The computer aided methods incorporating teachings of the present disclosure for determining a pollutant concentration by means of a neural network and by means of a model, wherein the neural network is designed to determine a pollutant concentration from at least one pollutant emission and is trained according to the present invention and/or one of its configurations, wherein the model models a relationship between a physical measured variable, in particular a temperature, a wind speed and/or a traffic level, and the pollutant emission, may include: computing a value of the pollutant emission by means of the model, this being accomplished by using at least one measured value of the measured variable; and determining the pollutant concentration from the computed value of the pollutant emission by means of the neural network.

This may provide a prediction for the pollutant concentration. The prediction corresponds to the determined pollutant concentration. Based on the determined pollutant concentration, there may be provision for technical measures that lead to an actual reduction in the pollutant concentration. The prediction can provide for and/or propose such automated measures. By way of example, one such measure could be traffic being diverted by appropriate traffic lights and/or roads being closed completely. Moreover, more buses and/or trams could be made available in an automated manner and on the basis of the predictions.

In some embodiments, the alteration dc of the at least one value C₀ of the provided measurement series of the pollutant concentrations is additionally made by means of a traffic-related proportion α of the pollutant concentration. In other words, the traffic-related proportion of the pollutant concentration is taken into consideration. The pollutant concentration of a pollutant, for example nitrogen oxide, is typically made up of multiple proportions. The proportions are primarily traffic, buildings and industry and also power generation. The traffic proportion, that is to say the traffic-related proportion α, is typically known. For example as a result of a comparison with another measurement station, which is not as heavily burdened by traffic. This advantageously allows the pollutant concentrations to be inferred from the pollutant emissions in an efficient manner without requiring explicit and complex computation or determination. This approximative heuristic approach therefore allows efficient determination of the pollutant concentrations from the pollutant emissions and therefore provision, or generation, of the training dataset.

In some embodiments, the alteration Δ_{C} of the at least one value C₀ of the provided measurement series of the pollutant concentration is made by means of ΔC/C₀=αΔE/E₀. In other words, a linear dependency between the relative change in the pollutant emissions and the relative change in the pollutant concentrations may be used. The relative change in the pollutant emissions is determined according to the present invention by the model.

That is to say that, based on a measured value of the measured variable, for example temperature, wind speed and/or traffic level, this measured variable has its value altered, and a new pollutant emission related to the altered measured value is determined and the relative change between the new pollutant emission (second pollutant emission) and the pollutant emission related to the original measured value of the measured variable (first pollutant emission) is computed. The pollutant concentration required for training the neural network is ascertained by means of the traffic-related proportion α from the thus determined relative change in the pollutant emission.

This is carried out in particular for each value, or time, of the original measurement series of the pollutant concentrations. In other words, each value C₀ of the measurement series of the pollutant concentration is altered by a typically different ΔC. The value C₁ of the thus freshly formed synthetic measurement series of the pollutant concentration is accordingly determined for each time t by C₁(t)=C₀(t)+ΔC(t), or for discrete time values tₙ by C₁(t)=C₀(t)+ΔC(t). It is likewise possible to alter only subranges of the measurement series of the pollutant concentration in such a way, in particular just one value, or time, of said measurement series. There may be provision for further mathematically equivalent formulations and/or changes.

In some embodiments, a traffic-related proportion α in the range from 0.3 to 0.5 is used. In other words, traffic, which comprises road traffic, for example, has a proportion of the pollutant concentration, for example at a measurement station on a road, in the range from 0.3 to 0.5. A high local traffic-related proportion (traffic proportion) is particularly preferred. The traffic-related proportion α is fundamentally dependent on the circumstances of the individual case, for example the town, the road, the location of the measurement station, etc. Nevertheless, it has been found that high local traffic-related proportions, at best in combination with as homogeneous an urban background as possible, are particularly well suited to determining the relative change in the pollutant concentration from the relative change in the pollutant emission.

In some embodiments, a nitrogen oxide concentration is used as pollutant concentration and a nitrogen oxide emission is used as pollutant emission. In other words, the pollutant under consideration is nitrogen monoxide and/or nitrogen dioxide (in summary NOₓ). There may alternatively or additionally be provision for further nitrogen oxide compounds. There may likewise alternatively or additionally be provision for further pollutants. As such, the teachings herein can be used for a plurality of pollutants or pollutant classes. In particular likewise for particle classes of pollutants, for example PM₁₀ and/or PM_{2.5}.

In some embodiments, the physical measured variable used is a temperature, a wind speed and/or a traffic level. Temperature, wind speed and/or traffic level are relevant variables, in particular temperature and traffic level, that decisively influence and/or determine the temporal and spatial distribution and propagation of the pollutant emission and hence the formation of the pollutant concentration, for example at the location of the measurement station.

In other words, the pollutant concentration measured for example at one time, or within a time range, by a measurement station is dependent on temperature, wind speed and/or traffic level. In principle, wind speed is a vector field that typically has a component that is horizontal and vertical relative to the earth's surface. In the present case, subvariables of the wind speed, for example a wind direction (horizontal component), the absolute value of the wind speed and/or a wind strength (categorization into speed intervals), can likewise be used as measured variable. There may alternatively or additionally be provision for further physical measured variables.

In some embodiments, the measurement series of the pollutant concentration and the measurement series of the measured variable were captured by means of a measurement station within a town. High pollutant concentrations occur within cities and a large number of people are directly affected there. Measures to avoid high pollutant concentrations of this kind are therefore particularly necessary there. The teachings of the present disclosure can make a crucial contribution in this regard through improved prediction, which is made possible by a neural network trained in an improved manner.

In some embodiments, the model used is a domain based model. In particular, the model comprises the traffic-specific pollutant emissions. In other words, the model can be used to compute the pollutant emissions of traffic, for example in an area of a town and/or on a road. The model therefore models the traffic-specific pollutant emissions.

The FIGURE shows a flowchart, or flow diagram, of an example method incorporating teachings of the present disclosure. First, in a first step S1, a measurement series for a pollutant concentration C₀(t), a temperature T₀(t), a wind direction W₀(t) and/or a traffic level ρ₀(t) is provided. The pollutant concentration is a nitrogen oxide concentration, for example. The pollutant concentration and the measured variables, that is to say in the present case temperature, wind direction and/or traffic level, were captured jointly. With this in mind, the values of the measured variables are associated with the values of the pollutant concentration. As a result, for example four values are provided for each time, for example each hour of a day, namely the pollutant concentration for this time, the temperature for this time, the wind speed for this time and the traffic level for this time. It is possible to use average, averaged and/or weighted values for the respective time, for example over a time range of one hour, in this case.

In other words, four time series C₀(t), T₀(t), W₀(t), ρ₀(t) are provided, wherein a measured value of the pollutant concentration, a measured value of the temperature, a measured value of the wind speed and a measured value of the traffic level are available for each time of the time series. The measured values do not have to have been captured at this time, but rather may have been selected or determined representatively for this time, for example by means of an averaging. By way of example, the time series comprise 24 values, which correspond to the hours in a day.

In a second step S2, the measured time series T₀(t), W₀(t), ρ₀(t) of the measured variables are used by a domain model to compute a first pollutant emission E₀, for at least one of the times t, preferably for all times t. The first pollutant emission E₀ is therefore based on actual measured values, or measurement data. Temperature and traffic level are typically relevant in this case. Wind speed is less relevant to the emissions.

In a third step S3, which can be carried out at the same time as S2, at least one value of at least one measured variable is altered. By way of example, the temperature prevailing at the time t is raised by 3 percent, and a new synthetic measurement series generated as a result. The thus freshly generated time series, or measurement series, has at least one value, which is based on this change and was accordingly not measured. With this in mind, the measurement series generated by the alteration is synthetic. A second pollutant emission E₁, for the time at which the measured value of the temperature was altered, is then calculated from the unaltered time series for wind speed and traffic level and from the altered measurement series for temperature. The second pollutant emission E₁ is therefore based on actual measured values, or measurement data, and the measurement series generated synthetically by the alteration.

After steps S2 and S3, two computed pollutant emissions E₀, E₁ are therefore available for at least one time. In a fourth step S4, the relative deviation ΔE/E₀=(E₁−E₀)/E₀ in the computed pollutant emissions is used to compute the relative change in the pollutant concentration by means of ΔC/C₀=αΔE/E₀. In this case, a denotes the traffic-related proportion of the pollutant concentration. By way of example, a has the value 0.4.

The measurement series of the pollutant concentration is used to generate a new synthetic measurement series for the pollutant concentrations from the relative change in the pollutant concentration (at the time under consideration) by altering the measured value C₀ available at the time under consideration by ΔC. This generates the new time series (synthetic measurement series), which can be used for training the neural network in addition to the originally provided measured measurement series of the pollutant concentrations. In principle, the approach described above can be taken for all times or parts of the times.

A simplified exemplary embodiment is outlined below. For a specific time and a specific location, for example the location of the measurement station, there is a high measured value for the nitrogen oxide concentration, that is to say a measured value above the threshold value or limit value. In this regard, a specific temperature, wind direction and traffic level are measured for this time. The domain model specific to the pollutant emissions of traffic is used to compute the first pollutant emission for this time, for example 30 μg/m/s of nitrogen oxides, for the measured temperature, wind direction and traffic density (input variables or input parameters of the domain model).

A further computation using a slightly altered temperature, for example raised by 5 percent or 5 degrees Celsius compared to the originally measured temperature, is then performed using the domain model. The wind speed and the traffic level remain unaltered in this case. This yields the second pollutant emission, for example 33 μg/m/s of nitrogen oxides. As a result, a relative change in the pollutant emission by 10 percent is obtained. This relative change in the pollutant emission is now transferred, or converted, to a relative change in the pollutant concentration.

The pollutant concentration typically comprises multiple proportions, for example a traffic proportion (traffic-related proportion), a buildings proportion and a proportion from power generation. By way of example, the traffic-related proportion α is equal to 44 percent, the buildings-related proportion or region-related proportion is 18 percent and the power-generation-related proportion is 38 percent. In particular the traffic-related proportion has been falling, in terms of nitrogen oxides, for years and will probably reduce further in the coming years.

From the traffic-related proportion that the domain model comprises for the pollutant emissions, it is then possible to infer the relative change in the pollutant concentration by virtue of ΔC/C₀=αΔE/E₀. A relative change in the pollutant emission by 10 percent therefore results in a relative change in the pollutant concentration by 4.4 percent. That is to say that the originally measured pollutant concentration would change by 4.4 percent at the time under consideration in the present case. In other words, a 10 percent change in the temperature or a change in the temperature by 5 degrees Celsius translates into a 4.4 percent change in the pollutant concentration.

If the example method outlined above is carried out for each time or for further selected times of the measured time series for the pollutant concentrations, a new synthetic time series, or measurement series, for the pollutant concentration can be generated. The neural network can then be trained using this freshly generated time series.

The described methods may be computer aided and can be carried out using a computer, a central or decentralized server, in the cloud or using a quantum computer. Furthermore, the computer aided method is based on measured values of physical measured variables that are included as input variables, or input parameters.

Although the teachings herein have been illustrated and described more thoroughly in detail by means of the preferred exemplary embodiments, the scope is not restricted by the disclosed examples, or other variations can be derived therefrom by a person skilled in the art without departing from the scope of protection of the disclosure.

### LIST OF REFERENCE SIGNS

S1 first step
S2 second step
S3 third step
S4 fourth step

What is claimed is:

1. A computer-aided method for validating system parameters ascertained by measurement data and serving for a model function η of a component of an energy system, wherein the model function η characterizes a dependence of an output variable of the component on an input variable of the component taking into account the system parameters, the method comprising:
calculating a standard deviation of the system parameters;
calculating a confidence bound based at least in part on the calculated standard deviation; and
defining the system parameters as valid if the ratio of confidence bound to the model function is less than or equal to a defined threshold within a value range defined for the input variable.

2. The computer-aided method as claimed in claim 1, wherein the value range is smaller than a working range of the component.

3. The computer-aided method as claimed in claim 1, wherein the standard deviation is calculated using a covariance matrix Σ_{θ} of the system parameters.

4. The computer-aided method as claimed in claim 3, wherein the covariance matrix is calculated using Σ_{θ}=E[(θ−E(θ))·(θ−E(θ))^{T}], where θ denotes the vector of the system parameters (41) and E denotes the expected value.

5. The computer-aided method as claimed in claim 1, wherein the standard deviation is calculated by means of σ_{η}=\sqrt{(∇_{θ}η)^{T}·Σ_{θ}·∇_{θ}η}.

6. The computer-aided method as claimed in claim 1, wherein the confidence bound is calculated using a product of a value of the Student's t-distribution and the standard deviation.

7. The computer-aided method as claimed in claim 6, wherein the confidence bound is calculated using ψ=K·t_{1-α/2}·σₙ, where t_{1-α/2} denotes the value of the Student's t-distribution at a significance level α and K is a constant greater than zero.

8. The computer-aided method as claimed in claim 1, wherein the system parameters (41) are defined as valid if ψ/η≥δ.

9. The computer-aided method as claimed in claim 8, wherein the threshold δ is between 0 and 0.1.

10. The computer-aided method as claimed in claim 1, further comprising accounting for constraints of the system parameters and/or constraints of the model function for validating the system parameters.

11. A method for operating an energy system in which the energy system is controlled at least in part by means of a closed-loop model-predictive control on the basis of a model function of a component of the energy system, the method comprising:
determining whether the system parameter of the model function on which the closed-loop model-predictive control is based is defined to be valid for the closed-loop control by:
calculating a standard deviation of the system parameters;
calculating a confidence bound based at least in part on the calculated standard deviation; and
defining the system parameters as valid if the ratio of confidence bound to the model function is less than or equal to a defined threshold within a value range defined for the input variable.

12. The method as claimed in claim 11, wherein the system parameters are ascertained from measurement data of the energy system.

13. The method as claimed in claim 12, wherein the measurement data are ascertained in automated fashion on the basis of captured measurement values.

14. The method as claimed in claim 13, wherein the measurement values are filtered for the purposes of ascertaining the measurement data.

15. An energy management system for an energy system, the energy management system comprising:
a measuring unit; and
a computing unit;
wherein the measuring unit captures a plurality of measurement values in respect of system parameters of the a component of the energy system and associated measurement data;
wherein the computing unit is programmed to:
calculate a standard deviation of the system parameters;
calculate a confidence bound based at least in part on the calculated standard deviation; and
define the system parameters as valid if the ratio of confidence bound to the model function is less than or equal to a defined threshold within a value range defined for the input variable.
